(19) United States
(12) Patent Application Publication
(10) Pub. No.: US 2008/0254118 A1
(43) Pub. Date: Oct. 16, 2008

(54) PROCESS FOR PREPARING PRAMIPEXOLE DIHYDROCHLORIDE TABLETS

(76) Inventor: Hans-Werner Wernersbach, Ingelheim (DE)

Correspondence Address: MICHAEL P. MORRIS;BOEHRINGER INGELHEIM USA CORPORATION, 900 RIDGEBURY ROAD, P O BOX 368, RIDGEFIELD, CT, 06877-0368, US

(21) Appl. No.: 11/734,041

(22) Filed: Apr. 11, 2007

(51) Int. Cl. A61K 9/20; A61K 31/428; A61P 25/16

(52) U.S. Cl. 424/464; 514/367

(57) ABSTRACT

The present invention relates to a process for preparing tablets of pramipexole dihydrochloride. In particular, the present invention relates to a process for preparing tablets of pramipexole dihydrochloride wherein the tablets exhibit enhanced storage stability properties.

### FIELD OF THE INVENTION

The present invention relates to a process for preparing tablets of pramipexole dihydrochloride. In particular, the present invention relates to a process for preparing tablets of pramipexole dihydrochloride wherein the tablets exhibit enhanced storage stability properties.

### BACKGROUND OF THE INVENTION

Pramipexole is a known dopamine D2 receptor agonist. It is structurally different from the ergot-derived drugs, e.g., bromocriptine or pergolide. It is also pharmacologically unique in that it is a full agonist and has receptor selectivity for the dopamine D2 family of dopamine receptors. Pramipexole was originally disclosed in U.S. Pat. Nos. 4,731,374, 4,843,086 and 4,886,812, all of which are incorporated herein by reference.

Pramipexole is designated chemically as (S)-2-amino-4,5,6,7-tetrahydro-6-(propylamino)benzothiazole and has the molecular formula C₁₀H₁₇N₃S and a relative molecular mass of 211.33. The chemical formula is as follows:

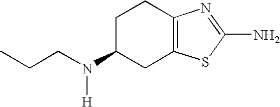

The solvate form commonly used is pramipexole dihydrochloride monohydrate (molecular formula C₁₀H₂₁Cl₂N₃OS; relative molecular mass 302.27). Pramipexole dihydrochloride monohydrate is a white to off-white, tasteless, crystalline powder. Melting occurs in the range of 296° C. to 301° C., with decomposition. Pramipexole is a chiral compound with one chiral center. The pure (S)-enantiomer is obtained from the synthetic process by chiral recrystallization of one of the intermediates during synthesis.

Pramipexole dihydrochloride monohydrate is a highly soluble compound. Water solubility is more than 20 mg/mL and solubility in buffer media is generally above 10 mg/mL between pH 2 and pH 7.4. Pramipexole dihydrochloride monohydrate is not hygroscopic, and has a highly crystalline nature. Under milling, the crystal modification (monohydrate) does not change. Pramipexole is very stable in the solid state, yet in solution it is light sensitive.

Pramipexole immediate release (IR) tablets were first authorized in the USA in 1997, followed over the course of the next few years by marketing authorizations in the European Union (EU), Switzerland, Canada, and South America as well as in countries in Eastern Europe, the Near East, and Asia.

Pramipexole IR tablets are indicated in the EU and US for the treatment of signs and symptoms of either early Parkinson's Disease or advanced Parkinson's Disease in combination with levodopa. The IR tablets are indicated to be taken 3 times a day.

The manufacturing process for pramipexole dihydrochloride monohydrate tablets, which was marketed in the USA in 2005 under the brand name MIRAPEX® (the marketed package/product hereinafter referred to as the “commercial formulation”), results in a tablet which has a relatively stable shelf life wherein approximately 95% of the labeled average amount of the active ingredient remains in the tablet after 18 months of storage. However, it is desirable to develop products having as close to zero degradation as possible upon being stored for extended periods of time.

The present invention relates to a process for preparing tablets of pramipexole dihydrochloride monohydrate wherein the tablets exhibit enhanced storage stability properties when compared to the commercial formulation.

### SUMMARY OF THE INVENTION

For purposes of this disclosure and invention, hereinafter the term “pramipexole dihydrochloride” means pramipexole dihydrochloride and the pharmaceutically acceptable solvates thereof in particular including the monohydrate of pramipexole dihydrochloride.

In accordance with the present invention, there is provided a process for producing tablets of pramipexole dihydrochloride wherein the tablets exhibit enhanced storage stability properties when compared to the commercial formulation. Compared to the commercial formulation, the pramipexole dihydrochloride tablets produced in accordance with the process of the invention exhibit a higher percentage of active ingredient remaining when stored under conventional storage conditions along with a decreased amount of degradation products.

Further provided is a process for preparing tablets of pramipexole dihydrochloride wherein the process involves formulating tablets comprising intra-granular tableting ingredients, pramipexole dihydrochloride, a binder and extra-granular tableting agents. The process comprises the steps of optionally sizing the intra-granular tableting ingredients to form substantially uniform sized particles of intra-granular tableting ingredients, forming a premix comprising the—optionally substantially uniform sized—intra-granular tableting ingredients, the pramipexole dihydrochloride and the binder, granulating the premix and drying said granulated premix to an endpoint moisture content of from about 1.0% to about 2.5% to form a dried premix, mixing the extra-granular tableting agents with the dried premix to form a final blend and compressing the final blend into tablets. In a further embodiment the granulated premix is dried to an end point moisture content of from about 1.5% to about 2.5%.

Further provided is a process for preparing pramipexole dihydrochloride tablets comprising intra-granular tableting ingredients, pramipexole dihydrochloride, a binder and extra-granular tableting agents, wherein at least a portion of the process is performed in a closed system and comprises the steps of:
(a) loading particles of the intra-granular tableting ingredients into a fluid bed granulator wherein the particles of the intra-granular tableting ingredients may optionally be sized prior to loading to form substantially uniform sized particles,
(b) dissolving the pramipexole dihydrochloride in water and povidone to form an aqueous pramipexole dihydrochloride solution and spraying the pramipexole dihydrochloride solution onto the particles of intra-granular tableting ingredients in the fluid bed granulator,
(c) preparing a binder suspension and adding the binder suspension to the fluid bed granulator by spraying,
(d) mixing the particles of intra-granular tableting ingredients, pramipexole dihydrochloride solution and binder suspension in the fluid bed granulator to form a premix,
(e) granulating said premix to form a granulated premix,
(f) drying said granulated premix to an endpoint moisture content of from about 1.0% to about 2.5%,
(g) mixing said granulated premix of step (f) with the extra-granular tableting agents and blending to form a final blend,
(h) compressing the final blend into tablets using a tablet press.

The tablets produced in accordance with the aforementioned process exhibit enhanced storage stability attributes when compared to the commercial formulation.

A further aspect of the invention includes a pharmaceutical tablet formulation comprising pramipexole dihydrochloride, wherein the average amount of pramipexole dihydrochloride remaining in the tablet at 18 months under storage conditions of 25° C. and a relative humidity of 60% is at least about 97% of the labeled amount.

Another aspect of the invention includes a pharmaceutical tablet formulation comprising pramipexole dihydrochloride, wherein the average amount of pramipexole dihydrochloride remaining in the tablet at 24 months under storage conditions of 25° C. and a relative humidity of 60% is at least about 95% of the labeled amount and further may be, preferably, at least about 97%.

An additional aspect of the invention includes a pharmaceutical tablet formulation comprising pramipexole dihydrochloride, wherein the average amount of total degradation product present in the tablet at 18 months under storage conditions of 25° C. and a relative humidity of 60% is less than about 1.0%.

The term “average amount” as used herein is calculated by determining the amount of the designated product (either active ingredient or degradation product) present in a particular sample of product and then taking an average of the samples of product.

Usually in the final commercial pramipexole product, the tablets are included as packaged products and packaging may include bottles, blister packs or the like.

These and other features, benefits and advantages of the invention will be apparent from the following disclosure.

### BRIEF DESCRIPTION OF THE DRAWINGS

FIG. 1 is a flow chart showing a process for producing pramipexole dihydrochloride tablets according to one aspect of the invention.

### DETAILED DESCRIPTION OF THE INVENTION

According to the present invention, pramipexole dihydrochloride tablets can be prepared which exhibit enhanced storage stability over the commercial formulation. This is valuable in the pharmaceutical arena as it enables pharmaceutical manufacturers to produce and store the pramipexole dihydrochloride tablets for longer periods thereby reducing concern as to whether the product has exceeded its useful life and requires disposal. This, in turn, enables pharmacies, and ultimately consumers, to enjoy the benefits of reduced costs associated with the need to monitor the efficacy of a product and the need to replenish the market supply due to expiration of the product.

In accordance with the invention, it has been found that by controlling certain parameters during the manufacture of pramipexole dihydrochloride tablets, the resulting tablets exhibit enhanced stability when compared to the commercial formulation. In particular, controlling the particle size of intra-granular tableting ingredients so that they possess a relative substantial uniformity (optional), preparation and use of a binder suspension, performing the process in a closed system, as well as controlling the moisture content of the product prior to tableting enables the production of a pramipexole dihydrochloride tablet which has highly desirable storage stability enhancements over the commercial formulation.

In accordance with the above, the pramipexole dihydrochloride tablets of the invention comprise intra-granular tableting ingredients, pramipexole dihydrochloride, a binder and extra-granular tableting agents. The process of the invention comprises the steps of sizing the intra-granular tableting ingredients to form substantially uniform sized particles of intra-granular tableting ingredients (optional step), forming a premix comprising the optionally uniformly sized intra-granular tableting ingredients, the pramipexole dihydrochloride and the binder, granulating the premix and drying said granulated premix to an endpoint moisture content (Loss on Drying (LOD) at 95° C.) of from about 1.0% to about 2.5% to form a dried premix, mixing the extra-granular tableting agents with the dried premix to form a final blend and compressing the final blend into tablets. In a further embodiment the granulated premix is dried to an end point moisture content of from about 1.5% to about 2.5%.

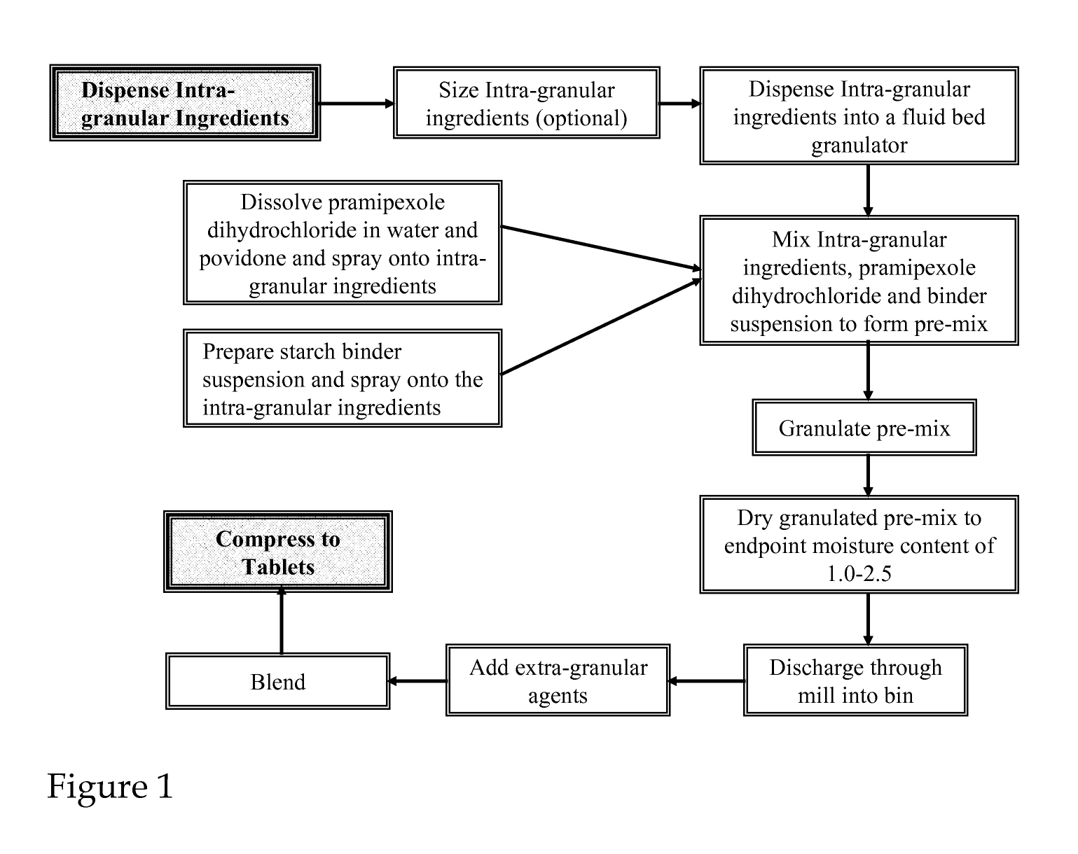

A process for formulating the tablets which may result in commercial pramipexole products of enhanced stability is set forth in FIG. 1. The process shown in FIG. 1 involves a process for preparing pramipexole dihydrochloride tablets comprising intra-granular tableting ingredients, pramipexole dihydrochloride, a binder and extra-granular tableting agents, wherein at least a portion of the process is performed in a closed system. The process comprises the steps of:
(a) loading particles of the intra-granular tableting ingredients into a fluid bed granulator wherein the particles of the intra-granular tableting ingredients may optionally be sized prior to loading to form substantially uniform sized particles,
(b) dissolving the pramipexole dihydrochloride in water and povidone to form an aqueous pramipexole dihydrochloride solution and spraying the pramipexole dihydrochloride solution onto the particles of intra-granular tableting ingredients in the fluid bed granulator,
(c) preparing a binder suspension and adding the binder suspension to the fluid bed granulator by spraying,
(d) mixing the particles of intra-granular tableting ingredients, pramipexole dihydrochloride solution and binder solution in the fluid bed granulator to form a premix,
(e) granulating said premix to form a granulated premix,
(f) drying said granulated premix to an endpoint moisture content of from about 1.0% to about 2.5%,
(g) mixing said granulated premix of step (f) with the extra-granular tableting agents and blending to form a final blend,
(h) compressing the final blend into tablets using a tablet press.

The intra-granular tableting ingredients include mannitol-D USP, colloidal silicon dioxide NF, povidone (K25) USP, corn starch NF and purified water USP.

The mannitol-D used as starting material in the process of the present invention preferably is of the delta crystal modification having a beta content of not more than 10%. In one embodiment, the beta contact is between 2.5 and 10%

The extra-granular tableting agents of the present invention include colloidal silicon dioxide NF, corn starch NF and magnesium stearate NF.

With respect to the intra-granular tableting ingredients and extra granular tableting ingredients, the following table represents the preferred amounts of tableting ingredients in each tablet as a percentage of the overall amount used in each batch as well as the amount of API (pramipexole dihydrochloride):

TABLE 1

| Ingredient | % per batch |
|---|---|
| Mannitol-D | 50-60 |
| Corn Starch | 35-45 |
| Colloidal Silicon Dioxide | 1-3 |
| Povidone | 1-3 |
| Magnesium Stearate | 1-3 |
| API | ** |

**The amount of API (pramipexole dihydrochloride) is dependent upon the desired tablet strength.

Tablet strengths can be from 0.125 mg to 1.5 mg with typical strengths being 0.125 mg, 0.25 mg, 0.50 mg, 0.75 mg, 1 mg, 1.5 mg and 2.0 mg.

The following table presents various tablet formulations which are representative, though not limiting, examples of tablet formulations according to the invention:

TABLE 2

| Component | mg/tablet | | | | | |
|---|---|---|---|---|---|---|
| API | 0.125 | 0.25 | 1.5 | 1.00 | 1.25 | 1.5 |
| Mannitol | 49.455 | 61.00 | 122.0 | 121.50 | 162.00 | 208.5 |
| Corn starch, | 25.010 | 30.90 | 61.8 | 61.85 | 82.55 | 106.0 |
| dried | | | | | | |
| Corn starch, | 7.300 | 9.00 | 18.0 | 18.00 | 24.00 | 30.8 |
| undried | | | | | | |
| Colloidal | 0.940 | 1.20 | 2.4 | 2.30 | 3.10 | 4.0 |
| Silicon | | | | | | |
| Dioxide | | | | | | |
| Povidone | 0.940 | 1.15 | 2.3 | 2.35 | 3.10 | 4.0 |
| (K25) | | | | | | |
| Magnesium | 1.230 | 1.50 | 3.0 | 3.00 | 4.00 | 5.2 |
| Stearate | | | | | | |
| Purified Water | * | * | * | * | * | * |
| Tablet Weight | 85.000 | 105.00 | 210.00 | 210.00 | 280.00 | 360.00 |

* For production, the purified water is adapted to the equipment used and does not appear in the final product.

The advantages to be realized from using the processes of the invention to produce the pramipexole dihydrochloride tablets of the invention include enhanced storage stability properties. Such enhanced storage stability properties include, but are not necessarily limited to, enhanced shelf life and decreased degradation products.

The enhanced shelf life of the pramipexole dihydrochloride tablets prepared according to the processes of the present invention is exhibited by the ability of the tablets to retain a higher percentage of active ingredient when stored under certain conditions compared to the commercial formulation stored under the same conditions.

In particular, the pramipexole dihydrochloride tablets prepared according to the process of the present invention has an average amount of pramipexole dihydrochloride remaining in the tablet at 18 months under storage conditions of 25° C. and a relative humidity of 60% of at least about 97% of the labeled amount. The commercial formulation stored under the same conditions average less than 95.8% ofthe labeled amount (average amount). The trend for the amount of active ingredient present in the stored tablets prepared according to the invention can be projected out to 24 and even 36 months where even at 36 months greater than 95% of the labeled amount should remain. This of course is significant as it allows for longer shelf life of the product and thus cost savings to consumers as the product does not have to be replaced by the manufacturer as frequently due to expiration of the unused product stored by the manufacturer, distributor and/or pharmacist.

A further advantage of the pramipexole dihydrochloride tablets prepared according to the process of the present invention involves the decreased amounts of degradation product which appear in the tablets upon storage.

Tablets prepared in accordance with the process of the invention contained lower total degradation product than compared with the commercial formulation. This is important in the pharmaceutical industry where purity of a pharmaceutical preparation is critical to the ultimate activity and stability of the formulation as well as the ultimate safety of the product.

The following Example is representative of the process used to prepare pramipexole dehydrate tablets according to the invention.

### EXAMPLE 2

The following process was used to prepare 0.5 mg tablets of pramipexole dihydrochloride:

Into a fluid bed granulator, the following intra-granular ingredients were dispensed while passing through a comil (Quadro) having a 1.4 mm screen:

| | |
|---|---|
| Mannitol-D: | 122,000 g |
| Colloidal Silicon Dioxide: | 1,200 g |
| Corn Starch: | 58,800 g |

In a separate stainless steel container, 500 g of pramipexole dihydrochloride monohydrate was dissolved in 20,000 ml of purified water with stirring and then 2,300 mg of polyvidone 25 (Povidone K25) was added and dissolved to completion with stirring. The pramipexole dihydrochloride solution was then sprayed onto the mixture of intra-granular ingredients in the fluid bed granulator. In a separate stainless steel container, 6,090 g of corn starch was added to 15,000 ml of purified water with stirring forming a starch paste. The starch paste was then added to 38,000 ml of purified water which had been heated to 95° C. and stirred at a rate of from about 350 RPM (stirring can be from about 250 RPM to about 1250 RPM). An additional 21,000 ml of purified water (room temperature) was then added and stirred at 350 RPM. The temperature was allowed to cool to about 60° C. (the temperature can be from about 55° C. to about 65° C. at this stage). The starch solution was then sprayed onto the intra-granular ingredients and pramipexole dihydrochloride mixture in the fluid bed granulator. The material in the fluid bed granulator was then granulated and dried to a residual moisture content of 2.3% to form a pramipexole raw granulate. An extra-granular blend of magnesium stearate (3,000 g), colloidal silicon dioxide (1,200 g) and corn starch (18,000 g) was mixed with 187,000 g of the pramipexole raw granulate in a Quadro comil equipped with a 1.1 mm sieve for 30 minutes at 10 RPM to form a final blend. The final blend was then compressed into tablets weighing 210 mg and containing 0.5 mg pramipexole dihydrochloride.

The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the appended claims.

What is claimed is:

1. A process for preparing pramipexole dihydrochloride tablets comprising intra-granular tableting ingredients, pramipexole dihydro chloride or a pharmaceutically acceptable solvate thereof, a binder and extra-granular tableting agents, wherein the process is performed in a closed system and comprises the steps of:
(a) loading particles of the intra-granular tableting ingredients into a fluid bed granulator,
(b) dissolving the pramipexole dihydrochloride or pharmaceutically acceptable solvate thereof in water and povidone to form an aqueous pramipexole dihydrochloride solution and spraying the pramipexole dihydrochloride solution to the particles of intra-granular tableting ingredients in the fluid bed granulator,
(c) preparing a binder solution and adding the binder solution to the fluid bed granulator,
(d) mixing the particles of intra-granular tableting ingredients, pramipexole dihydrochloride solution and binder solution in the fluid bed granulator to form a premix,
(e) granulating said premix to form a granulated premix,
(f) drying said granulated premix to an endpoint moisture content of from about 1.0% to about 2.5%,
(g) mixing said granulated premix of step (f) with the extra-granular tableting agents and blending to form a final blend,
(h) compressing the final blend into tablets using a tablet press.

2. The process of claim 1 further comprising the step of sizing the intra-granular tableting agents prior to loading to a substantially uniform size.

3. The process of claim 1 wherein pramipexole dihydrochloride monohydrate solvate is used.

4. The process of claim 1 wherein the binder solution is an aqueous suspension comprising corn starch.

5. The process of claim 1 wherein the intra-granular tableting ingredients comprise mannitol-D, colloidal silicone dioxide, and corn starch.

6. The process of claim 1 wherein the extra-granular tableting agents comprise colloidal silicon dioxide, starch and magnesium stearate.

7. The process of claim 5 wherein the mannitol-D has no more than 10% beta modification product present.

8. A product produced in accordance with the process of claim 1.

9. A pharmaceutical tablet formulation comprising pramipexole dihydrochloride or a pharmaceutically acceptable solvate thereof, wherein the average amount of pramipexole dihydrochloride or pharmaceutically acceptable solvate thereof remaining in the tablet at 18 months under storage conditions of 25° C. and a relative humidity of 60% is at least about 97% of the labeled amount.

10. The tablet formulation of claim 9 wherein the pramipexole dihydrochloride is pramipexole dihydrochloride monohydrate solvate.

11. A pharmaceutical tablet formulation comprising pramipexole dihydrochloride, wherein the average amount of pramipexole dihydrochloride remaining in the tablet at 24 months under storage conditions of 25° C. and a relative humidity of 60% is at least about 95% of the labeled amount.

12. The tablet formulation of claim 11 wherein the pramipexole dihydrochloride is pramipexole dihydrochloride monohydrate solvate.

13. The pharmaceutical tablet of claim 11 wherein the average amount of pramipexole dihydrochloride is at least about 97% of the labeled amount.

14. A pharmaceutical tablet formulation comprising pramipexole dihydrochloride or a pharmaceutically acceptable solvate thereof, wherein the average amount of total degradation product present in the tablet at 18 months under storage conditions of 25° C. and a relative humidity of 60% is less than about 1.0%.
